(19) United States
(12) Patent Application Publication
(10) Pub. No.: US 2004/0191258 A1
(43) Pub. Date: Sep. 30, 2004

(54) Prevention and treatment of the porcine reproductive and respiratory syndrome (PRRS) using immunoglobulins obtained from egg yolk from hens hyperimmunized with the PRRS virus

(76) Inventors: Jose Andres Morales Garzon, Tehuacan (MX); Eduardo Lucio Decanini, Tehuacan (MX)

Correspondence Address: Kristen R. Paris, Locke Liddell & Sapp LLP, Suite 2200, 2200 Ross Avenue, Dallas, TX, 75201-6776, US

(21) Appl. No.: 10/695,104

(22) Filed: Oct. 28, 2003

(30) Foreign Application Priority Data
Oct. 30, 2002 (MX) PA/A/2002/011761

(51) Int. Cl. A61K 39/42; C07K 16/08

(52) U.S. Cl. 424/159.1; 530/388.3

(57) ABSTRACT

The instant invention relates to the use of immunoglobulins obtained from the egg yolk of hens hyperimmunized with PRRS virus. The immunoglobulins are obtained through the extraction of the aqueous phase of the yolk through the use of hydroxypropylmethylcellulose phtalate at a final concentration of 0.05% and sodium azide at a final concentration of 0.001%. The invention also relates to the administration of these immunoglobulins for the prevention and treatment of pigs infected with the PRRS virus in order to lower mortality rates, obtain weight gain and diminish viral excretion in the herds.

### FIELD OF THE INVENTION

The instant invention relates to a new method for the treatment and prevention of porcine reproductive and respiratory syndrome (PRRS) based on the parenteral administration of immunoglobulins obtained from egg yolk from hens hyperimmunized with the PRRS virus.

### BACKGROUND OF THE INVENTION

The Porcine Reproductive and Respiratory Syndrome (PRRS) is a serious illness affecting pigs, which was reported in the United States of America in 1987 and was then identified in various other European countries. In 1991, Holland reported the isolation of the etiological agent called Lelystad virus and because of the symptoms presented by the pigs, it was known as the Porcine Epidemic Abortion and Respiratory Syndrome.

There are two ways of protecting animals against infectious agents: they can exposed to antigens derived from an infectious agent to stimulate a protective immunitary reaction or they can receive a preformed antibody obtained from an immune subject. The first way is conducted through different types of vaccines: freeze-dried live viruses or bacteria, dead viruses o bacteria in oily emulsions; and recently the creation of cloned and recombinant vaccines. Each of them presents advantages and drawbacks with regard to protection, immune response and protection duration. Besides, in some cases, there are undesirable lesions in the host because of the vaccine virus (Tizard, I. R. 1998)

The second form of protection, also called passive immunity, includes the transmission of antibodies specific against infectious agents in a host.

Traditionally, at research level, the antibodies are mainly obtained in mammals and less frequently in birds. The types antibodies obtained are monoclonal and polyclonal antibodies in mammals and polyclonal antibodies in birds (Larsson, et al. 1993).

In the case of birds, the chicken is the only species from which antibodies are obtained in a most accessible and highly defined form. The main serum antibody present in the chicken is IgG even though IgG is transported to the egg in a way similar to the transfer of mammal IgG through the placenta. In the egg, IgG is found in higher concentrations in the yolk, although it is also found in small concentrations in the albumin; it is even found in larger quantities in the yolk than in the hen serum (Larsson, et al. 1993).

To have an idea of the quantity of antibodies made in the hen, we must take into account that an egg-laying hen produces approximately 5 to 6 eggs per week with a yolk volume of about 15 ml. Thus, in a week, a hen produces antibodies in yolk equivalent to 90-100 ml of serum or 180-200 ml of whole blood. This is to be compared with the 20 ml of whole blood given per week by an immunized rabbit. Obviously if we use animals such as horses or cows, the quantity of serum and antibodies is larger than in the egg but it is more expensive and more painful for the animals. Among the advantages of the antibodies found in the yolk of hen egg, we can mention the following ones:

1.—They do not fix the complement

2.—They do not bind to the Protein A of Staphilococcus aureus

3.—They do not react with the Rheumatoid Factor

4.—Because of its phylogenetic difference with mammal antibodies, the IgG does not cross react with the mammal antibodies.

5.—Low cost.

Recently, egg yolk antibodies (immunoglobulins) have been employed as tools for diagnostic and therapy (Schmidt, et al. 1989). Thus, taking advantage of its phylogenetic difference with mammal immunoglobulins, the Ig's have presented several advantages when used in immune diagnosis. For example, yolk Ig's have been used to detect several viruses through ELISA, immunodiffusion, immunofluorescence and complement fixing. Because of its low isoelectric point, compared to human Ig, they are employed in electrophoresis assays for the quantification of immunoglobulins in the serum of several animals (Altschuh, D. 1984, Larsson, et al. 1988, Larsson, et al. 1992, Larsson, et al. 1993, Schade, R. 1996). With regard to their therapeutic application, the Ig's have been used as immunotherapy in several scientific fields. For example, the administration of egg yolk immunoglobulins orally has prevented rotavirus infections in mice, bovines, and pigs, among others (Ikemori, et al 1992, Kuroki, et al 1994, Marquardt, et al 1998). Moreover, they have been used as antivenins against viper and scorpions, that can be injected to neutralize the toxins without the risk of anaphylactic reactions commonly caused by antivenins elaborated in horse (Larsson, et al. 1993). A further application has been to prevent caries caused by Streptococcus mutans in humans (Hatta, H. et al 1984).

Objects, Uses and Advantages of the Instant Invention

The object of the instant invention is to offer a prevention and treatment method for PRRS. Through the parenteral administration of immunoglobulins specifically directed against the causal agent, obtained from the egg yolk of hens hyperimmunized with one or several PRRS viruses and the subsequent demonstration of their presence in the blood of the treated animals.

Another object of the instant invention is to foment weight increase in animals treated with immunoglobulins specifically directed against PRRS.

Moreover, within the instant invention, the use of inmmunoglobulins obtained form egg yolk against PRRS is claimed to eliminate or substantially reduce the signology and mortality, transmission and prevention of PRRS virus in treated animals.

Finally, the invention relates to a process to prepare a product based on immunoglobulins obtained from egg yolk specifically directed against PRRS.

Through the practice of the instant invention, the dissemination of the PRRS causing virus diminishes; moreover, the productive parameters of the animals improve. The immunoglobulins obtained are administered parenterally in aqueous solution, through deep injection.

### DETAILED DESCRIPTION OF THE INVENTION

The detailed characteristics of this novel invention are obvious in the description hereinafter and in the enclosed figures.

The instant invention is based on the fact that the immunoglobulins extracted of the aqueous phase of the egg yolk grant protection against viral and bacterial illnesses.

Hen Immunization Program

To obtain the immunoglobulins (Igs) specifically directed against PRRS, it is necessary to have a vaccination schedule in a flock of SPF (specific Pathogens Free) type birds.

The vaccination schedule is made in the following way: a dose (0.5 ml) consisting of an emulsioned water in oil type vaccine (70% oil and 30% water) containing an inactivated PRRS virus, is administered subcutaneously to each one of the hens, 8 weeks of age, in the mid posterior third part of the neck. The complete vaccination program included 2 boosts, at a 4-week interval with regard to the last vaccination; that is to say, at 12 and 16 weeks of age.

Extraction of Yolk Immunoglobulins

There are different methods to extract Igs from egg yolk. In the instant invention, we used the Yokoyama method (Yokoyama, H. et al 1993) with the modification that Avid AL was not used.

In short, the process was as follows. The extraction of antibodies from the yolk was made in two steps. In the first step, the yolk was diluted 1:8 (without albumin) with 0.0001% sodium azide and stored under refrigeration during at least 24 hours. Then, the supernatant was separated and then 5% hydroxypropylmethylcellulose phtalate (HPMCP) was added in proportion of 0.25 ml for every 100 ml of yolk. It was allowed to rest during at least 24 hours and the lipid layer formed in the upper part of the solution was separated. It was filtered and bottled. The quality control tests include:

1.—Sterility test (to check that the product is free from bacterial, fungi and yeast contamination according to the Code of Federal Regulations of the United States of America.

2.—Quantification of antibodies against PRRS. The technique of micro virus serum neutralization, beta method (dilution of constant virus sample) is used, on 96-well microplates, flat bottom and MA104 cell growth. The immunoglobulins are diluted from 1:40 to 1:10240 on the microplate using medium 199 as diluent, 200 DICT 50 (infective dose in tissue culture) of PRRS virus are added, incubation at 37° C. during 30 minutes and transfer of the mixture to a monolayer of MA104 cells of 24 hours of incubation, then incubation during 4-5 days at 37° C. and 5% CO₂. A title from 1:40 on is considered satisfactory.

Hereinafter, tests are presented as non-limitative examples. Said tests show the use of immunoglobulins against PRRS in piglets object of the instant invention.

### EXAMPLE 1

Three sows, 50 days of age, weighing about 20 kg, were placed in 2×2 m pens, and individually identified. One of them received a dose of Ig against PRRS (5 ml) equivalent to a 0.4 ml per kg of body weight dose, intramuscularly. Another sow received twice the dose of Ig against PRRS (10 ml) through the same route. The third sow is a control animal, without treatment. Before the immunoglobulin application, the three sows were bled to determine the antibodies against PRRS through the MNT test for PRRS. During 4 weeks after the treatment, the sows were bled and the antibody levels caused by the immunoglobulins were determined through the MNT test against PRRS in MA 104 cells. The lesions at the site of application were also assessed as well as any sign suggesting the presence of the illness.

FIG. 1 presents the results obtained in the immunized sows. It can be seen that with the two treatments using immunoglobulins, high levels of antibodies against PRRS were obtained in the first week after the treatment and then a notable diminution is seen, but the levels are still higher than in the control sow. This is an indication of the half life and shows that the antibodies supplied by the instant invention remain in the blood flow during three weeks.

### EJEMPLO 2

Four hundred and fifty-two piglets weighing about 7 kg were used and administered Igs dose intramuscularly, repeating the dose two weeks after the first administration. On the other hand, 420 control piglets were not administered a treatment. The evaluated parameters were weight gain, the virus presence through PCR and ELISA test for PRRS and mortality percentage.

Table 1 shows the parameters of weight gain and mortality in both groups. It was observed that the group treated did not show weight gain as was expected compared to the control group, but a reduction in mortality percentage was seen in the group treated with immunoglobulin. In the same way, the PCR test shows posivity in the control group from the fourth week on, while in the group treated with immunoglobulins, positivity was seen from ninth week on after the immunoglobulin treatment.

Table 1. Differences in the different parameters between the group treated with immunoglobulins against PRRS versus control group.

| | Group treated with | |
|---|---|---|
| | Immunoglobulins | Control Group |
| Number of Animals | 452 | 420 |
| Initial weight | 6.17 | 6.05 |
| Final weight | 26.55 | 30.55 |
| Mortality | 19 | 53 |
| Mortality % | 4.20 | 12.62 |

FIG. 2 presents the results of the ELISA test with serums of treated and controlled pigs. The results show a lower exposition of the pigs to the infectious agent in the group treated with immunoglobulins compared to the control group, in which the presence of the virus was detected since the fifth week. FIG. 2 also shows the mortality of the treated pigs, the serology obtained and the weight gain.

### BRIEF DESCRIPTION OF THE FIGURES

FIG. 1. Shows the determination of antibodies in the serum of pigs treated with two different doses of immunoglobulins administered intramuscularly.

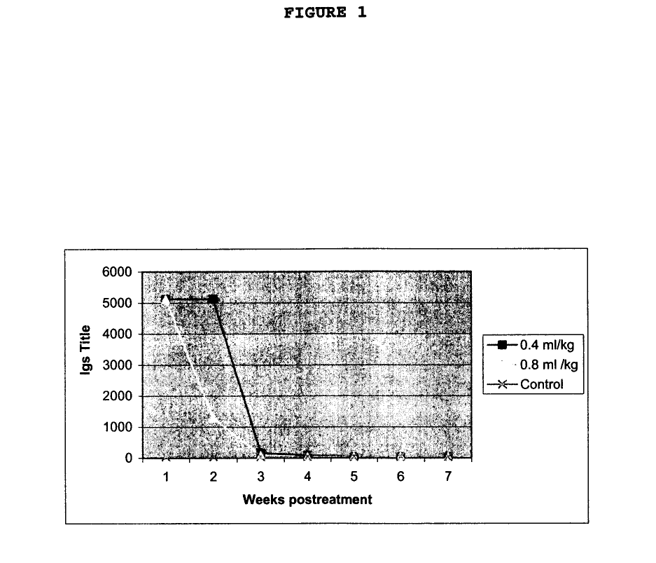

FIG. 2. Shows the results of presence of antibodies against PRRS measured by the ELISA test in treated and control sows.

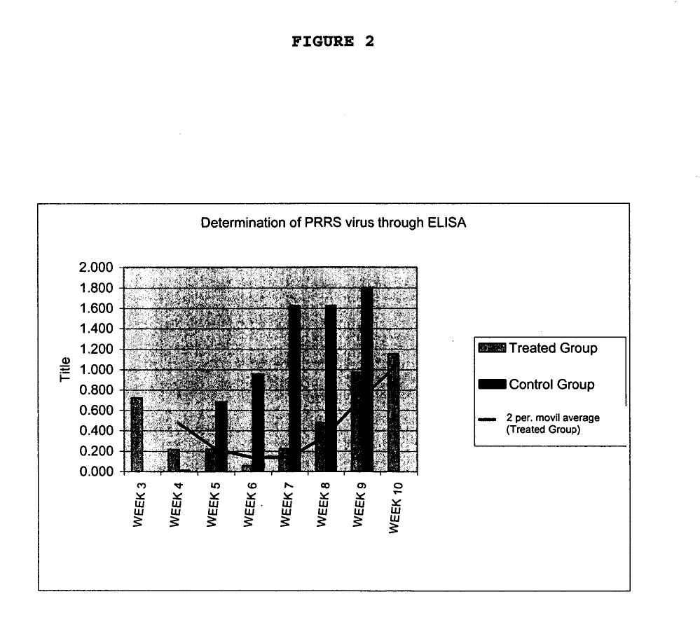

### Bibliografía

Altschuh, D. et al. 1984. Determination of IgG and IgM levels in serum by Rocket Immunoelectrophoresis using yolk antibodies from Immunized chickens. J.Immunolog.Methods. 69:1-7

Hatta, H. et al. 1997. Passive Immunization Against Dental Plaque Formation in Humans: Effect of a Mouth Rinse containing Egg Yolk Antibodies (IgY) Specific to Streptococcus mutans. Caries.Res.31:268-274.

Ikemori, Y. et al. 1992. Protection of neonatal calves against fatal enteric colibacillosis by administration of egg yolk powder from hens immunized with k99-pillated enterotoxigenic Escherichia coli. Am.J.Vet.Res.53:2005-2008.

Kuroki, M. et al 1994. Passive protection against bovine rotavirus in calves by specific immunoglobulins from chicken egg yolk. Arch.Virol. 138: 143-148.

Larsson, A. et al. 1988. Chicken antibodies: a tool to avoid false positive results by rheumatoid factor in latex fixation tests. J.Immunol.Methods. 108:205-208.

Larsson, A. et al. 1992. Chicken antibodies: a tool to avoid interference by complement activation in ELISA. J. Immunol. Methods. 156: 79-83.

Larsson, A. et al. 1993. Chicken antibodies: taking advantage of evolution. A review. Poultry Sci.72: 1807-1812.

Marquart, R. 1998. Antibody-loaded eggs for piglets: prevention of mortality of baby pigs from diarrhea. Proc. 2^{nd} International Symposium on Egg Nutrition and Newly Emerging Ovo-Technologies. Alberta, Canada.

Schade, R. et al 1996. The production of avian (Egg yolk) antibodies:IgY. Atla.24:925-934.

Tizard, I. R. 1998. Vacunación y vacunas In: Inmunología Veterinaria. 5^{a} Ed. Mc Graw-Hill. pp285-305.

Yokoyama, H. et al. A two step procedure for purification of hen yolk immunoglobulin G: Utilization of Hydroxypropylmethylcellulose phtalate and synthetic affinity ligand gel (Avid AL®). Poultry Sci. 72:275-281.1993.

1. The use of immunoglobulins for the treatment of pigs infected with PRRS virus.

2. The immunoglobulins of claim 1, obtained through the exhaustive vaccination of egg-laying hens SPF type with an inactivated PRRS vaccine.

3. The immunoglobulins of claim 2, obtained through the extraction of the aqueous phase of egg yolk.

4. The immunoglobulins of claim 3, obtained through the use of 0.001% sodium azide and 5% hydroxypropylmethylcellulose phtalate, and whose antibody title for PRRS should not be lower than 1:80 per each 0.050 ml.

5. The immunoglobulins of claim 4, in which the quantity used for the PRRS treatment in pigs should not be lower than 0.4 ml per kg of weight, administered intramuscularly.

6. The immunoglobulins of claim 5, absorbed and then found in the blood flow of the treated animals.

7. The immunoglobulins of claim 6 offer protection against PRRS virus when they are administered every 2 weeks intramuscularly.

8. The immunoglobulins of claim 7 lower the mortality in treated pigs.
